(19) United States
(12) Patent Application Publication
(10) Pub. No.: US 2015/0196372 A1
(43) Pub. Date: Jul. 16, 2015

(54) Method and Device for Preparing the Fitting of a Dental Implant

(71) Applicant: 3DKreaForm, Vaulx-en-Velin (FR)

(72) Inventor: Guillaume Champleboux, La Murette (FR)

(21) Appl. No.: 14/418,584

(22) PCT Filed: Jun. 4, 2013
(86) PCT No.: PCT/FR2013/051268

§ 371 Date: Jan. 30, 2015

(30) Foreign Application Priority Data
Jul. 31, 2012 (FR) 1257404

(51) Int. Cl. A61C 8/00; A61B 6/14; A61B 19/00; A61C 9/00

(52) U.S. Cl. CPC A61C 8/0089; A61C 9/0053; A61C 9/008; A61B 6/14; A61B 19/54; A61B 2019/5437

(57) ABSTRACT

The invention relates to method of preparing the fitting of at least one dental implant, comprising the linking of a first three-dimensional model of the external surface of at least a part of the buccal cavity of a patient or of a reproduction made of a solid material of said at least one part of the buccal cavity and of a second three-dimensional model of at least a part of the bone system of the jaw of the patient on the basis of the recognition of faces of a benchmark element in the first model and of at least one portion of the benchmark element in the second model.

### BACKGROUND

The present invention relates to the fitting of dental implants intended to hold prostheses in place.

### Discussion of the Related Art

When a patient's teeth are strongly degraded, it may be envisaged to replace the missing teeth with dental prostheses. The prosthesis may be anchored in the patient's upper or lower maxillary via an implant or a plurality of implants screwed in the jaw. The fitting of an implant thus requires drilling a hole in the jaw, the implant being then screwed in this hole.

A difficulty lies in the fact that, when a patient's teeth are strongly degraded, the bone system of his/her jaws often also is in a bad condition. The locations where implants can be implanted are thus strongly limited and should be determined with a great accuracy.

A conventional method for assisting the fitting of implants comprises forming a stent made of transparent resin, also called radiological guide, which is the negative reproduction of the patient's jaw. The forming of the stent generally requires taking a material impression of the jaw to be implanted, casting a plaster copy from this impression, a prosthetic study by a practitioner during which radio-opaque false teeth or radio-opaque markers maybe placed on the plaster copy and the stent is designed based on the plaster copy containing the radio-opaque false teeth or markers. The radio-opaque false teeth or markers indicate the desired positions of the hole drilling axes for the screwing of implants. It is then verified that these positions are compatible with the bone system of the jaw. For this purpose, images of the bone system are obtained, for example, by CT scanning while the patient has the stent in his/her mouth. Once the positions of the implants are accurately defined, cylindrical openings may be drilled in the stent to install metal tubes. Such tubes guide the tool with which the holes are drilled in the jaw during the implant fitting. The stent thus modified is called drilling guide or surgical guide.

The determination of the real positions by means of the images of the bone system is performed more or less empirically by the practitioner, by looking at the bone system images taken when the patient has the stent in his/her mouth. This is a delicate operation which requires a highly-trained practitioner. Further, accurately determining the position of the implants may be difficult.

It would be desirable to assist the practitioner in the determination of the real positions of the implants. It would further be desirable to be able to accurately determine the positions of the implants.

The method of preparation to the fitting of implants is long. Indeed, it requires taking the impression of the patient's jaw, forming the plaster copy, forming the stent based on the plaster copy, and eventually acquiring images with an X-ray scanner while the stent is in the patient's mouth. To form the drilling guide, the stent should further be machined in accordance with the definition of the position of the implants such as virtually positioned by means of the CT scan images. Further, the method is constraining for the patient since it is necessary for the patient to be present a first time so that the impression of his/her jaw can be taken, a second time after the stent has been formed, so that the scanner images can be acquired, and finally a third time on the day of the surgery. It would be desirable to decrease the duration of the process of preparing the fitting of a dental implant and to require the patient's presence a smaller number of times.

### SUMMARY

Thus, an embodiment of the present invention aims at at least partly overcoming the disadvantages of previously-described methods for preparing the fitting of implants.

The present invention aims at providing a method for preparing the fitting of dental implants which facilitates and improves the accuracy of the determination of the position of the implants.

Another object of an embodiment of the present invention is to be able to form a surgical guide by means of a computer-assisted manufacturing tool.

Another object of an embodiment of the present invention is that it is not necessary to take a material impression of the patient's jaw.

Another object of an embodiment of the present invention is that the preparation method requires the patient's presence only once.

To achieve this, an aspect of an embodiment of the invention provides a method of preparing the fitting of at least one dental implant, comprising the steps of:

fixing at least one fiducial element to at least a portion of a patient's oral cavity;

acquiring, by means of an optical sensor or of a touch probe, data relative to said at least a portion of the oral cavity;

delivering a first three-dimensional model of the external surface of said at least a portion of the oral cavity based on data relative to said at least a portion of the oral cavity;

acquiring, by means of an X-ray scanner, data relative to at least a portion of the bone system of the patient's jaw;

delivering a second three-dimensional model of said at least a portion of the bone system of the patient's jaw based on data relative to said at least a portion of the bone system of the jaw;

performing a computer recognition of faces of said at least one fiducial element in the first model;

determining by computer means a first three-dimensional coordinate system associated with the first model;

performing a computer recognition of at least a portion of said at least one fiducial element in the second model;

determining by computer means a second three-dimensional coordinate system associated with the second model;

determining by computer means a transformation relationship for passing from the first coordinate system to the second coordinate system based on said faces and on said portion; and

determining the position of said implant based on the first model and on the second model and based on the first coordinate system, on the second coordinate system, and on the transformation relationship.

According to an embodiment of the present invention, the step of determining the position of said implant based on the first model and on the second model and based on the first coordinate system and on the second coordinate system comprises the steps of:

determining the position of a dental prosthesis associated with said implant in the first model;

determining the theoretical position of the implant in the second model based on the position of the associated dental prosthesis in the first model; and

determining the ideal position of the implant in the second model based on the theoretical position.

According to an embodiment of the present invention, the method further comprises the steps of:

fixing an additional fiducial element to another portion of the oral cavity opposite to said portion;

acquiring, by means of the optical sensor or of the touch probe, data relative to said other portion of the oral cavity;

delivering a three-dimensional model of the external surface of said other portion of the oral cavity based on data relative to said other portion of the oral cavity;

acquiring, by means of the optical sensor or of the touch probe, data relative to the fiducial element and to the additional fiducial element when the mouth is in occlusion; and

delivering a three-dimensional model of the external surface of the fiducial element and of the additional fiducial element when the mouth is in occlusion.

According to an embodiment of the present invention, the step of determining the position of said implant is carried out based on the first model and on the three-dimensional model of the external surface of said other portion of the oral cavity placed in occlusion.

According to an embodiment of the present invention, a denture is capable of being placed on said portion of the oral cavity, the first model being determined in the absence of the denture, the method further comprising the steps of:

arranging the denture on said portion of the oral cavity;

acquiring, by means of the optical sensor or of the touch probe, data relative to the denture; and

delivering a three-dimensional model of the external surface of the denture based on the data relative to the denture.

According to an embodiment of the present invention, the method further comprises determining the drilling axis of said implant, determining a three-dimensional model of a stent comprising a cylindrical opening along the drilling axis of said implant, and manufacturing in computer-assisted fashion said stent comprising said opening.

According to an embodiment of the present invention, the step of determining the second coordinate system comprises determining the inertia matrix of said portion.

According to an embodiment of the present invention, the method further comprises the steps of:

fixing at least three fiducial elements to said portion of a patient's oral cavity;

performing a computer recognition of faces of each fiducial element in the first model;

determining by computer means, for each fiducial element, a first reference point in the first model;

performing a computer recognition of at least one portion of each fiducial element in the second model;

determining by computer means, for each fiducial element, a second reference point in the second model; and

determining by computer means the transformation relationship for passing from the first coordinate system to the second coordinate system based on the first three reference points and the second three reference points.

Another aspect of an embodiment of the present invention provides a system for preparing the fitting of dental implants, the system comprising:

at least one fiducial element comprising at least three non-parallel faces visible with an optical camera and/or a touch probe and at least a portion locatable with X rays, said fiducial element being capable of being fixed to at least a portion of a patient's oral cavity;

an optical image sensor or a touch probe capable of acquiring data relative to said at least a portion of the oral cavity;

an X-ray scanner capable of acquiring data relative to at least a portion of the bone system of the patient's jaw; and

a processing unit connected to the X-ray scanner and to the optical image sensor and/or to the touch probe, the processing unit, the optical image sensor, and/or the X-ray scanner being capable of delivering a first three-dimensional model of the external surface of said at least a portion of the oral cavity based on the data relative to said at least a portion of the oral cavity, delivering a second three-dimensional model of said at least a portion of the bone system of the patient's jaw based on the data relative to said at least a portion of the bone system of the jaw, recognizing the faces in the first model, determining a first three-dimensional coordinate system associated with the first model, recognizing said at least a portion in the second model, determining a second coordinate system associated with the second model, determining a transformation relationship for passing from the first coordinate system to the second coordinate system based on said faces and on said portion, and determining the position of said implant based on the first model and on the second model and based on the first three-dimensional coordinate system, on the second coordinate system, and on the transformation relationship.

According to an embodiment of the present invention, said faces are made of a material transparent to X rays.

According to an embodiment of the present invention, the material transparent to X rays is further opaque to visible light.

According to an embodiment of the present invention, said portion corresponds to an insert.

According to an embodiment of the present invention, at least two faces out of said three faces are planar and inclined with respect to each other by an angle preferably in the range from 5° to 85° or from 95° to 270°.

According to an embodiment of the present invention, at least one of the faces corresponds to a portion of a sphere or to a portion of a cylinder.

According to an embodiment of the present invention, said portion is covered with said faces.

According to an embodiment of the present invention, said portion comprises at least two rectilinear non-intersecting tubes.

According to an embodiment of the present invention, said portion comprises at least three spheres having non-aligned centers.

According to an embodiment of the present invention, the spheres have different diameters.

According to an embodiment of the present invention, said portion comprises at least one parallelepiped.

According to an embodiment of the present invention, the fiducial element comprises at least first, second, and third non-parallel planar faces, at least the first face being inclined with respect to the second face by an angle preferably in the range from 5° to 85° or from 95° to 270° and the first face being inclined with respect to the third face by an angle preferably in the range from 5° to 85° or from 95° to 270°.

Another aspect of an embodiment of the present invention provides a system for preparing the fitting of dental implants for implementing the method such as previously defined, the system comprising:

a fiducial element such as previously defined;

an optical image sensor and/or a touch probe;

an X-ray scanner; and

a processing unit connected to the X-ray scanner and to the optical image sensor and/or to the touch probe.

### BRIEF DESCRIPTION OF THE DRAWINGS

The foregoing and other features and advantages will be discussed in detail in the following non-limiting description of specific embodiments in connection with the accompanying drawings, among which:

FIGS. 1 and 2 are simplified perspective views of an embodiment of a fiducial element according to the invention;

FIGS. 3 to 11 are simplified perspective views of other embodiments of the fiducial element according to the invention;

FIG. 12 shows the fiducial element of FIG. 1 glued to a patient's tooth;

FIG. 13 shows the fiducial element of FIG. 11 fixed to a patient's jaw;

FIG. 14 partially and schematically shows an embodiment according to the invention of a system for preparing the fitting of implants;

FIG. 15 shows in the form of a block diagram an embodiment according to the invention of a method of preparing the fitting of implants;

FIG. 16 shows in the form of a block diagram a variation of the embodiment according to the invention of the method of preparing the fitting of implants illustrated in FIG. 15; and

FIG. 17 illustrates, in the form of a block diagram, another variation of the embodiment according to the invention of the method of preparing the fitting of implants illustrated in FIG. 15.

For clarity, the same elements have been designated with the same reference numerals in the various drawings and, further, the various drawings are not to scale.

### DETAILED DESCRIPTION

Unless otherwise indicated, in the following description, expressions “approximately”, “substantially”, and “in the order of” mean “to within 10%”.

A known method of preparing the fitting of implants comprises the following step sequence:

(1) Taking an impression of the jaw to be implanted by means of an impression material such as silicon, an alginate, a hydrocolloid, etc.

(2) Casting a plaster copy from this impression.

(3) Estimation by the practitioner, based on the impression, of the ideal positions of the prostheses.

(4) Forming a transparent resin stent based on the plaster copy. The resin stent contains either radio-opaque false teeth, or radio-opaque markers, for example, cylinders or cones, made of a material visible with X rays such as gutta-percha, which represent the desired positions of the axes of the implants associated with the ideal positions of the prostheses. The stent is the negative reproduction of this copy with which it should be able to intimately fit. All the faces of the stent which do not come into contact with the plaster copy have an arbitrary shape. The stent provided with the radio-opaque false teeth or with the radio-opaque markers is called radiological guide. It then has to be determined whether these estimated drilling axes, which are ideal from a prosthetic viewpoint, are compatible with the bone structure of the jaw.

(5) Performing an X-ray scanner examination of the patient having the radiological guide in his/her mouth.

(6) Determination by the practitioner, based on the image of the radio-opaque markers, whether each implant can be placed at the desired location while respecting the various endoosseous elements, and according to which approximate trajectory. If the implant cannot be placed at the desired location, the practitioner estimates the displacement to another position with respect to the marker included in the radiological guide.

(7) Possibly, drilling the stent, which will then be used as a surgical guide for the drilling of the jaw at the location where the implant is desired to be inserted.

The principle of the invention is to determine the position of the implants by using both a three-dimensional modeling of the teeth and of the bone structure of the patient's jaw where the soft tissues, particularly the gums, are not shown (or at least are not sufficiently identifiable), and a three-dimensional modeling of the inside of the patient's mouth, obtained directly or indirectly, where the surface of the soft tissues, particularly the gums, as well as the surface of the existing teeth, are shown.

The three-dimensional modeling of the bone structure can be obtained by computer tomography based on images delivered by an X-ray scanner. The three-dimensional model of the bone structure and of the teeth is called three-dimensional internal model hereafter.

The three-dimensional surface modeling of the inside of the patient's mouth may be obtained from images provided by an intraoral sensor, for example, an optical camera, capable of being introduced into the patient's mouth or based on images (particularly in the form of a three-dimensional cloud of points) provided by an optical three-dimensional surface sensor or by a three-dimensional touch probe by using a reproduction made of a solid material of the portion of the oral cavity to be modeled, particularly when the practitioner has no intraoral camera. The three-dimensional model of the external surface of the soft tissues (particularly the gums) and of the teeth of a patient's mouth is called three-dimensional external model hereafter.

The joint use of the three-dimensional internal model and of the three-dimensional external model enables the practitioner to place the implants more easily and accurately. In particular, the soft tissues may be represented, for example, as superposed to the bone system of the jaw on a same display device.

Further, when a surgical guide is to be formed, a three-dimensional model of the entire external surface of the surgical guide can be determined. The surgical guide can then be assisted by computer-assisted manufacturing tools.

Further, when the three-dimensional external model is determined by using an intraoral camera introduced into the patient's mouth, steps (1) to (4) of the previously-described method of preparing the fitting of implants and resulting in the forming of a radiological guide are then no longer necessary. The duration of the method of preparing the fitting of implants can thus be decreased.

The position of the implants is determined by linking the three-dimensional external model which contains specific and distinct information helping the positioning of the prosthesis and the three-dimensional internal model which contains information relative to the bone structure of the jaw. The positioning of the implants and the linking of the external and three-dimensional internal models are performed by using a computer.

According to an embodiment of the present invention, the linking of the three-dimensional external model and of the three-dimensional internal model is performed by using a fiducial element present in the patient's mouth on acquisition of the images used to determine the three-dimensional external model and the three-dimensional internal model and which is at least partly visible on the three-dimensional external model and on the three-dimensional internal model.

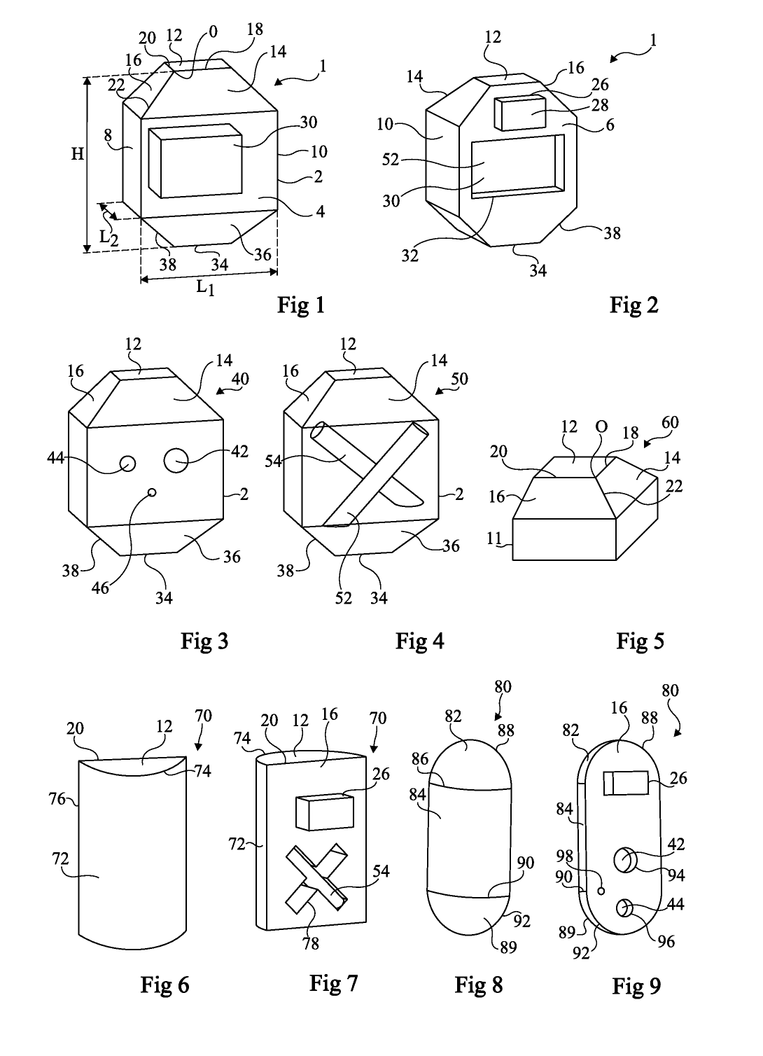

FIGS. 1 and 2 schematically show two perspective views of an embodiment of a fiducial element 1 according to the invention. Fiducial element 1 comprises a block 2 having the general shape of a central cuboid comprising two opposite ends extending in a truncated pyramid. Block 2 has a height H in the range from 5 mm to 50 mm, a length L₁ in the range from 5 mm to 50 mm, and a width L₂ in the range from 4 mm to 40 mm.

Block 2 comprises a front face 4, a rear face 6, two lateral faces 8, 10. Front and rear faces 4 and 6 are planar and parallel and lateral faces 8 and 10 are planar, parallel to each other, and perpendicular to faces 4, 6.

At a first end, fiducial element 1 comprises three external fiducial faces 12, 14, 16 which are used to recognize fiducial element 1 in the three-dimensional external model. Fiducial faces 12, 14, and 16 of fiducial element 1 are made of a material substantially opaque to visible light, to be visible in images obtained by an optical image acquisition device. It further is a material which produces no artifacts on images acquired by an optical camera as well as on CT images obtained by an X-ray scanner. As an example, it is polyatheratherketone (PEEK) or polyoxymethylene (POM). It further is a material compatible with the temporarily placing of fiducial element 1 in a patient's mouth.

In the present embodiment, fiducial faces 12, 14, 16 are non-parallel planar faces. Preferably, faces 12 and 14 have a common edge 18, faces 12 and 16 have a common edge 20, and faces 14 and 16 have a common edge 22. Preferably, the three edges 18, 20, and 22 join at a point O. As a variation, faces 12 and 14 may be connected to each other in a rounded portion. This may also apply for faces 12 and 16 and/or for faces 14 and 16.

In the embodiment shown in FIG. 1, face 14 is inclined with respect to face 12 by an angle of approximately 45°. Face 16 is inclined with respect to face 12 by an angle of approximately 45° and face 16 is inclined with respect to face 14 by an angle of approximately 45°.

Generally, face 12 is inclined with respect to face 16 by an angle which may preferably vary from 5° to 270°, preferably from 5° to 85° or from 95° to 270°. Face 14 is inclined with respect to face 16 by an angle which may preferably vary from 5 to 270°. Face 12 is inclined with respect to face 14 by an angle which may preferably vary from 5° to 85° or from 95° to 270°.

As shown in FIG. 2, fiducial element 1 comprises an outgrowth 26, comprising a face 28, preferably planar, which projects from face 6. As a variation, outgrowth 26 is not present. In operation, fiducial element 1 is intended to be temporarily placed in a patient's mouth. To achieve this, face 28 of fiducial element 1 may be temporarily glued to a tooth or to the patient's gum.

Fiducial element 1 comprises, in addition to fiducial faces 12, 14, and 16, additional fiducial faces 34, 36, 38. As an example, faces 34, 36, 38 correspond to the symmetrical images of faces 12, 14, 16 with respect to a plane of symmetry. Generally, face 34 is inclined with respect to face 38 by an angle which may preferably vary from 5° to 90°. Face 36 is inclined with respect to face 38 by an angle which may preferably vary from 5° to 90° or from 95° to 270°. Face 34 is inclined with respect to face 36 by an angle which may preferably vary from 5° to 90° or from 95° to 270°.

Fiducial element 1 comprises one or a plurality of radio-opaque inserts 30. Radio-opaque inserts means an insert substantially opaque to X rays.

A characteristic of radio-opaque inserts is that they are made of a material visible with X rays to be locatable by a scanner. The radio-opaque insert or inserts are selected to be made of a material which produces no artifacts during the scanning, which leaves a sufficiently contrasty mark on the scanner image (which for example corresponds, in the images delivered by the scanner, to pixels having a variable grey level) with respect to tissues or bones, and which has a sufficient mechanical resistance. The radio-opaque insert(s) are for example made of titanium or of aluminum.

Preferably, fiducial element 1, except for the radio-opaque insert(s), is entirely made of a material which is substantially transparent to X rays. Thereby on acquisition of an image delivered by a scanner, only the radio-opaque insert(s) of fiducial element 1 appear clearly in the image. In particular, fiducial faces 12, 14, and 16 do not substantially appear in the three-dimensional internal model. In the present embodiment, radio-opaque insert 30 is housed in an opening 32 provided in fiducial element 1. As a variation, the material opaque to visible light may be overmolded on the radio-opaque insert(s).

As a variation, for certain embodiments, the fiducial element may be entirely made of a radio-opaque material. In this case, faces 12, 14, 16 are made of a material substantially opaque to X rays.

In the embodiment shown in FIG. 1, the radio-opaque insert comprises a parallelepiped 30 of a radio-opaque material housed in an opening 32 which emerges on face 6 of fiducial element 1. To facilitate the understanding of the present invention, insert 30 is shown in full line in FIG. 1 while it is actually hidden by block 2. It may be a cuboid 30. As an example, the radio-opaque insert of fiducial element 1 only comprises parallelepiped 30.

FIG. 3 shows a fiducial element 40 according to another embodiment of the invention. Fiducial element 40 differs from fiducial element 1 in that parallelepipedal radio-opaque insert 30 is replaced with three spheres 42, 44, and 46 made of a radio-opaque material and, for example, embedded in the bulk of fiducial element 40. To make the present invention more easily understandable, spheres 42, 44, and 46 are shown in full lines in FIG. 3 while they are actually hidden by block 2. The centers of spheres 42, 44, and 46 are not aligned. Preferably, the spheres have different diameters. The diameter of sphere 44 may be smaller than the diameter of sphere 42 and the diameter of sphere 46 may be smaller than the diameter of sphere 44. As an example, the radio-opaque inserts of fiducial element 40 only comprise the three spheres 42, 44, and 46.

FIG. 4 shows a fiducial element 50 according to another embodiment of the invention. Fiducial element 50 differs from fiducial element 1 in that parallelepipedal radio-opaque insert 30 is replaced with two radio-opaque tubes 52, 54. As an example, tubes 52, 54 are embedded in the bulk of block 2, even though, to ease the understanding of the present invention, they are however shown in full line in FIG. 4. The two tubes 52, 54 may be solid rectilinear tubes 52, 54, having their respective axes, for example, contained in two planes parallel to each other and substantially perpendicular to the occlusal plane when fiducial element 50 is placed in the patient's mouth. Tubes 52 and 54 are for example embedded in the material forming body 2 of fiducial element 50 during the manufacturing process thereof.

The axes of tubes 52 and 54 are not parallel to each other and form, for example, an angle in the range from 30 to 120°, preferably 90°. As shown in further detail hereafter, the function of tubes 52 and 54 is to define two non-intersecting straight lines in images reconstructed based on CT scan slices.

It should be noted that tubes 52 and 54 are such that their respective axes do not intersect. However, the embodiment shown in FIG. 4, where the axes of tubes 52 and 54 are in parallel planes, is a preferred arrangement of tubes 52 and 54 since it minimizes the bulk of fiducial element 50 to enable to place fiducial element 50 in a patient's mouth.

FIG. 5 shows a fiducial element 60 corresponding to a variation of fiducial element 1 shown in FIG. 1 where fiducial faces 34, 36, and 38 are not present.

FIGS. 6 and 7 show two perspective views of an embodiment of a fiducial element 70 according to another embodiment of the invention. Fiducial element 70 differs from fiducial element 1 in that faces 16 (and 38) and 6 are confounded and in that face 14 (and 36) is replaced with a face 72 corresponding to a half-cylinder. Preferably, faces 12 and 72 have a common edge 74 corresponding to a half-circle and faces 12 and 16 have a common edge 20 corresponding to a line segment. Fiducial element 70 further comprises tubes 52, 54 (only tube 54 being shown in FIG. 7) of a radio-opaque material housed in an opening 78.

FIGS. 8 and 9 show two perspective views of an embodiment of a fiducial element 80 according to another embodiment of the invention. Fiducial element 80 differs from fiducial element 1 in that faces 16 (and 38) and 6 are confounded and in that face 12 is replaced with a face 82 corresponding to a quarter of a sphere and in that face 14 is replaced with a face 84 (and 36) corresponding to a half-cylinder. Preferably, faces 82 and 84 have a common edge 86 corresponding to a half-circle and faces 82 and 16 have a common edge 88 corresponding to a half-circle. Further, fiducial face 36 is replaced with face 89 which corresponds to the symmetrical image of face 82 with respect to a plane of symmetry and corresponds to a quarter of a sphere. Preferably, faces 89 and 84 have a common edge 90 corresponding to a half-circle and faces 89 and 16 have a common edge 92 corresponding to a half-circle. Fiducial element 80 further comprises spheres 42, 44, and 46 (only spheres 42 and 44 being shown in FIG. 9) of a radio-opaque material housed in openings 94, 96, 98.

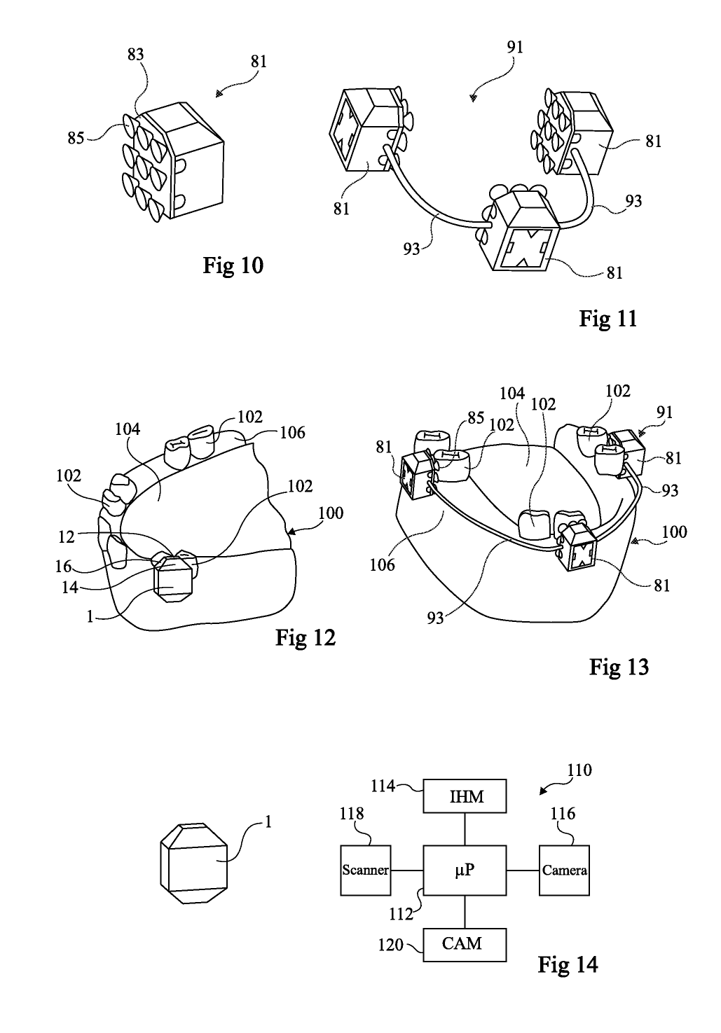

FIG. 10 shows a fiducial element 81 corresponding to a variation of fiducial element 60 where a fastening element 83 is provided at the rear face of fiducial element 81. Fastening element 83 comprises suction cups 85. As an example, nine suction cups 85 are shown in FIG. 10. Fastening element 83 is preferably made of a material substantially transparent to X rays. Suction cups 85 enable to fix fiducial element 81 in a patient's oral cavity without using glue or an adhesive material. Fastening element 83 may be provided with all the embodiments of fiducial elements previously described in relation with FIGS. 1 to 9.

FIG. 11 shows a fiducial device 91 which comprises three unit fiducial elements which may each correspond to one of the fiducial elements previously described in relation with FIGS. 1 to 10. As an example, in FIG. 11, fiducial device 91 comprises three fiducial elements 81 such as shown in FIG. 10. One of unit fiducial elements 81 may be connected by a flexible wire 93 to each of the two other unit fiducial elements. Wires 93 are preferably made of a material which creates no artifact in X-ray images. Wires 93 are for example made of titanium.

FIG. 12 partially and schematically shows oral cavity 100 of a patient. It shows teeth 102, tongue 104, and gums 106 of a patient. As described in further dental hereafter, on implementation of the method for preparing the fitting of implants according to the invention, fiducial element 1 shown in FIG. 1 is fixed to a tooth 102 at the level of face 28 of outgrowth 26. As an example, element 1 may be glued to a tooth 102 or to gum 106 by means of glue or of an adhesive material which provides a temporary gluing. The glue is for example based on a stone paste. Preferably, fiducial element 1 is glued so that the three previously-described faces 12, 14, and 16 can be easily seen in images acquired by an optical camera displaced in the patient's mouth by a practitioner. Preferably, fiducial face 12 is placed to be substantially parallel to the occlusal plane in the patient's mouth. Preferably, radio-opaque insert(s) 30 are placed substantially under the tooth 102/gum 106 line so that artifacts likely to be created by metal parts, such as dental amalgams or bridges, have no influence on the detection of radio-opaque markers on the CT images delivered by the X-ray scanner.

FIG. 13 partially and schematically shows oral cavity 100 of FIG. 12 having fiducial device 91 of FIG. 11 fixed thereto. Each unit fiducial element 81 of fiducial device 91 is fixed to a tooth 102 or to gum 106 via suction cups 85. As a variation, the unit fiducial elements of fiducial device 91 may be fixed to teeth 102 or to gum 106 by means of glue or of an adhesive material which provide a temporary bonding. The positioning of each fiducial element 81 may follow the conditions previously described in relation with FIG. 12. Advantageously, one unit fiducial element 81 is fixed to each lateral portion of the jaw, the third unit fiducial element 81 being capable of being fixed to the front part of the jaw.

FIG. 14 partially and schematically shows an embodiment according to the invention of a system 110 for preparing the fitting of implants. System 110 comprises a processing unit 112 (μP) connected to a man-machine interface 114 (IHM), to an optical and/or tactile analysis unit 116, and to an X-ray analysis unit 118. Processing unit 112 may further be connected to a computer-assisted manufacturing unit 120 (CAM). Processing unit 112 may correspond to a computer for example comprising at least a microcontroller and a memory. Man-machine interface 114 may comprise a display, which may be a touch screen, a keyboard, a mouse, etc. System 110 further comprises a fiducial element 1. As a variation, the fiducial element may correspond to any of previously-described fiducial elements 40, 50, 60, 70, 80, or 81 or to previously-described fiducial device 91.

Processing unit 112 is capable of linking the three-dimensional external model to the three-dimensional internal model.

According to an embodiment of the invention, optical and/or tactile analysis unit 116 comprises an intraoral optical camera capable of acquiring images from a patient's oral cavity. According to another embodiment of the invention, analysis unit 116 comprises an optical camera or a three-dimensional touch probe capable of acquiring images of objects outside of the oral cavity. Analysis unit 116 is capable of transmitting the obtained images to processing unit 112. Processing unit 112 is capable of determining the three-dimensional external model based on the images delivered by analysis unit 116.

According to an embodiment of the invention, X-ray analysis unit 118 comprises a scanner capable of acquiring X-ray images of a patient's oral cavity. X-ray analysis unit 118 is capable of transmitting the obtained images to processing unit 112. Processing unit 112 is capable of determining the three-dimensional internal model based on the images delivered by X-ray analysis unit 118.

According to an embodiment of the invention, optical analysis unit 116 comprises a device for delivering a three-dimensional external model of the patient's oral cavity, for example, the device commercialized by “3M ESPE” under name “Scanner intra-oral lava S.O.S.”. According to another embodiment of the invention, analysis unit 116 comprises a device for delivering a three-dimensional external object model, for example, the device commercialized by Straumann under name 3D Etkon, which uses a video camera, or the device commercialized by Renishaw under name Scanner Piccolo, which uses a 3-axes mechanical touch probe. Analysis unit 116 is capable of transmitted the three-dimensional external model to processing unit 112.

According to an embodiment of the invention, analysis unit 118 comprises an X-ray tomography device, for example, a CTCB (Cone Beam Computerized Tomography) device. Unit 118 is capable of determining the three-dimensional internal model of the patient's oral cavity and of transmitting the three-dimensional internal model to processing unit 112.

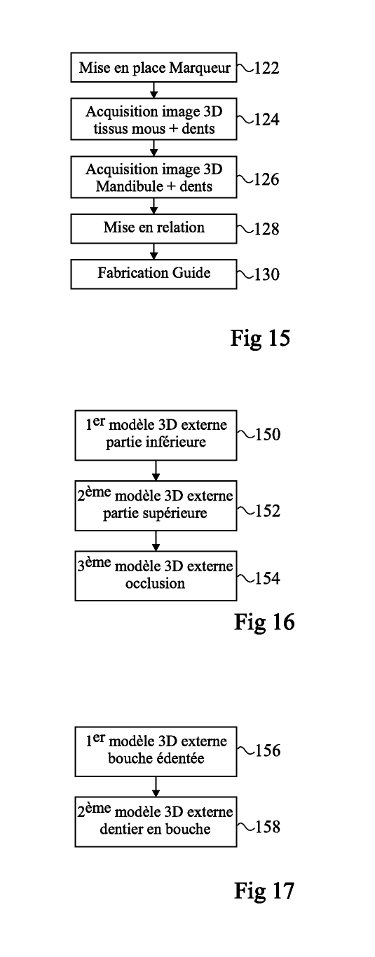

FIG. 15 shows a block diagram illustrating an embodiment of a method of preparing the fitting of dental implants according to the invention which may be implemented with system 110 described in FIG. 14 and particularly with any of previously-described fiducial elements 1, 40, 50, 60, 70, 80, 81 or with fiducial device 91.

At step 122, the dentist places fiducial element 1 or fiducial device 91 in a patient's mouth. Fiducial element 1 or fiducial device 91 may be temporarily fixed, for example, via a glue, to a tooth 102 or a plurality of teeth 102 as shown in FIG. 12 or 13 or to gum 106 of the patient. When the fiducial element corresponds to fiducial element 81, fiducial element 81 may be fixed to a tooth 102 or to gum 106 of the patient via suction cups 85. Fiducial element 1 or fiducial device 91 is then fixed with respect to the patient's lower or upper jaw for at least the duration of the next steps 124 and 126. The method carries on at step 124.

At step 124, the three-dimensional external model is determined, while fiducial element 1 or fiducial device 91 is present in the patient's mouth. The three-dimensional external model may be determined by processing unit 112 based on the images delivered by camera 116. For this purpose, the practitioner at least partially inserts camera 116 into the patient's mouth and acquires images of the external surface of teeth 102, of gums 106, and of fiducial element 1 or of fiducial device 91, camera 116 being displaced in the patient's mouth during the image acquisition. As a variation, the three-dimensional external model may be determined by optical analysis unit 116. The three-dimensional external model corresponds, for example, to a file of points or to a file at the STL format, which is a format frequently used for stereolithography software. A powder may be spread in the patient's mouth to decrease the occurrence of artifacts on the images acquired by camera 116. The method carries on at step 126.

At step 126, the three-dimensional internal model is determined while fiducial element 1 or fiducial device 91 is present in the patient's mouth. The three-dimensional internal model may be obtained by X-ray tomography. Data, for example, two-dimensional images, may be delivered by X-ray scanner 118 and the three-dimensional internal model may be determined by processing unit 112 from these data by means of a tomographic reconstruction algorithm. The two-dimensional images may be at the DICOM (Digital Imaging and Communications in Medecine) format. As an example, a set of two-dimensional images is obtained along equally distributed cross-section planes (for example, one image every millimeter), each image substantially having the same number of regularly-distributed pixels. The three-dimensional internal model then corresponds to a volumetric grid defined by all these images. Each volume element of the grid, or voxel, is assigned a digital value, for example, representative of a quantity of absorbed X radiation, for example obtained from the values of the pixels which surround the voxel. As a variation, the three-dimensional internal model may be determined by X-ray analysis unit 118. The method carries on at step 128. Steps 124 and 126 may be carried out in any order.

At step 128, processing unit 112 links the three-dimensional external model delivered at step 124 and the three-dimensional internal model delivered at step 126. This may be carried out as follows. Processing unit 112 determines a first three-dimensional reference system, also called first three-dimensional coordinate system, associated with the three-dimensional external model, having the location of the elements of the three-dimensional external model determined relative thereto, and a second three-dimensional reference system, also called second three-dimensional coordinate system, associated with the three-dimensional internal model, having the location of the elements of the three-dimensional internal model determined relative thereto. Each reference system may be determined by an origin, also called reference point hereafter, and three vectors. As an example, for fiducial elements 1, 40, 50, 60, 70, 80, and 81, the first reference system is determined from the analysis of the portion of the three-dimensional external model which corresponds to fiducial element 1, 40, 50, 60, 70, 80, or 81. More specifically, processing unit 112 determines the first reference system based on the recognition of the fiducial faces of the fiducial element which are present in the three-dimensional external model. As an example, for fiducial elements 1, 40, 50, 60, 70, 80, and 81, the second reference system is determined based on the analysis of the portion of the three-dimensional internal model which corresponds to fiducial element 1, 40, 50, 60, 70, 80, or 81. Processing unit 112 determines the second reference system based on the recognition of radio-opaque insert(s) 30, 42, 44, 46, 52, or 54 present in the three-dimensional internal model. For fiducial elements 1, 40, 50, 60, 70, 80, 81 or fiducial device 91, processing unit 112 then determines the transformation for passing from the first reference system to the second reference system that is, the three-dimensional geometric transformation which links the first and second reference systems. As an example, for fiducial elements 1, 40, 50, 60, 70, 80, and 81, the transformation is determined from the relative position of the fiducial faces and the radio-opaque inserts, which is known according to the design of the fiducial element.

As an example, in the case of fiducial element 1 shown in FIG. 1, the algorithm for determining the first reference system associated with the three-dimensional external model may comprise the steps of:

determining planar fiducial faces 12, 14, and 16 by means of a surface recognition algorithm;

determining edge 18 common to faces 12 and 14, edge 20 common to faces 12 and 16, and edge 22 common to faces 14 and 16;

determining the origin of the first reference system which corresponds to point O common to edges 18, 20, and 22; and

determining three non-coplanar vectors defining the first reference system, two vectors being provided by edges 18 and 20 and the third vector being equal to the vectorial product of the two previously-defined vectors.

As a variation, in the algorithm for determining the first reference system associated with the previously-described three-dimensional external model, face 4 may be used instead of face 12. Further, since previously-described fiducial element 1 has a symmetrical structure, fiducial faces 34, 36, and 38 may be used instead of faces 12, 14, and 16.

The use of fiducial element 1 shown in FIG. 1 may be advantageous since fiducial faces 12, 14, and 16 are inclined by 45° with respect to one another. Indeed, this decreases the risk for one of fiducial faces 12, 14, 16 not to be visible in the images acquired by optical camera 116 as compared with fiducial faces which would be inclined with respect to one another by a 90° angle. As an example, in the case of fiducial element 70 shown in FIGS. 6 and 7, the algorithm for determining the first reference system associated with the three-dimensional external model may comprise the steps of:

determining fiducial faces 12, 72, and 16 by means of a surface recognition algorithm;

determining edge 74, corresponding to an arc of a circle, common to faces 12 and 72 and determining the center of this arc of a circle which corresponds to the origin of the first reference system;

determining edge 20, corresponding to a line segment, common to faces 12 and 16, the center of arc of a circle 74 being located on segment 20; and

determining three non-coplanar vectors defining the first reference system, the first vector corresponding to edge 20, the second vector being perpendicular to the first vector, passing through the point of origin and being contained in face 12, the third vector being equal to the vectorial product of the previously-defined first and second vectors.

As an example, in the case of fiducial element 80 shown in FIGS. 8 and 9, the algorithm for determining the first reference system associated with the three-dimensional external model may comprise the steps of: determining fiducial faces 82, 84, and 16 by means of a surface recognition algorithm;

determining edge 86, corresponding to an arc of a circle, common to faces 82 and 84, determining the center of this arc of a circle which corresponds to the origin of the first reference system, and determining the plane containing this arc of a circle;

determining the intersection of face 16 and of the plane containing arc of a circle 86, which provides a segment having the center of arc of a circle 86 located thereon; and

determining three non-coplanar vectors defining the first reference system, the first vector corresponding to the segment of intersection of face 16 and of the plane containing arc of a circle 86, the second vector being perpendicular to the first vector, passing through the origin and being contained in the plane containing arc of a circle 86, the third vector being equal to the vectorial product of the first and second previously-defined vectors.

In the case of fiducial element 1 shown in FIG. 1 which comprises a radio-opaque parallelepiped 30, the algorithm for determining the second reference system associated with the three-dimensional internal model may comprise the steps of:

determining all the voxels of the three-dimensional internal model belonging to radio-opaque insert 30;

determining the center of gravity of radio-opaque insert 30;

determining the inertia matrix of radio-opaque insert 30; and

determining the eigenvectors of the inertia matrix.

The second reference system is defined by the center of gravity of radio-opaque insert 30 and the three eigenvectors of the inertia matrix of radio-opaque insert 30.

Generally, the shape of radio-opaque insert 30 may be different from that of a parallelepiped as long as it is capable of enabling to determine distinct and unique eigenvectors of the inertia matrix of the insert.

As an example, in the case of fiducial element 40 shown in FIG. 3, the algorithm for determining the second reference system associated with the three-dimensional internal model may comprise the steps of:

determining all the voxels of the three-dimensional internal model belonging to spheres 42, 44, 46;

determining centers of spheres 42, 44, 46;

determining a first vector corresponding to the vector connecting a first center (for example, the sphere of greatest diameter) to a second center (for example, the sphere of inter-mediate diameter) and a second vector corresponding to the vector connecting the first center to the third center (for example, the sphere of smallest diameter);

determining a third vector from the vectorial product of the first and second vectors; and

determining the origin of the second reference system corresponding to one of the centers.

As an example, in the case of fiducial element 50 shown in FIG. 4, the algorithm for determining the second reference system associated with the three-dimensional internal model may comprise the steps of:

determining all the voxels of the three-dimensional internal model belonging to radio-opaque inserts 52, 54;

determining the axes of tubes 52, 54;

determining the point at a minimum distance between the two axes of tubes 52, 54 which corresponds to the origin of the second reference system;

determining a first vector corresponding to the direction vector of the axis of tube 52 and a second vector corresponding to the direction vector of the axis of tube 54; and

determining a third vector from the vectorial product of the first and second vectors.

As an example, when fiducial device 91 is used, the first reference system, the second reference system, and the transformation for passing from the first reference system to the second reference system may be determined as follows.

The first reference system may correspond to the specific reference system of the three-dimensional external model used by optical and/or tactile analysis unit 116 and/or processing unit 112 during the delivery of the three-dimensional external model. The second reference system may correspond to the specific reference system of the three-dimensional internal model used by X-ray analysis unit 118 and/or processing unit 112 during the delivery of the three-dimensional internal model.

As an example, in the case where fiducial device 91 is used, the transformation for passing from the first reference system to the second reference system may comprise the steps of:

determining a first reference point for each unit fiducial element 81 in the three-dimensional external model. The first reference point may correspond to the origin of the first reference system which is determined in the case where fiducial element 81 is used alone and which may be determined according to the previously-described embodiments. The coordinates of each first point are expressed in the first reference system;

determining a second reference point for each unit fiducial element 81 in the three-dimensional internal model. The second reference point may correspond to the origin of the second reference system which is determined in the case where fiducial element 81 is used alone and which may be determined according to the previously-described embodiments. The coordinates of each second point are expressed in the second reference system;

determining a third reference point for each unit fiducial element 81 in the second reference system. For each fiducial element 81, the third reference point of the fiducial element corresponds to the first reference point having its coordinates expressed in the second reference system. This may be obtained by applying a translation to the second reference point, the applied translation vector being known according to the design of unit fiducial element 81; and

determining based on the first reference points and on the third reference points the transformation for passing from the first reference system to the second reference system. This transformation may be determined according to the algorithm described in the publication entitled “Least-Squares Fitting of Two 3-D Points Sets” by K. S. Arun, T. S. Huang, and S. D. Blostein (IEEE Transactions On Pattern Analysis And Machine Intelligence, Vol. PAMI-9, n° 5, pp. 698-700, September 1987).

Generally, to implement this algorithm, an integer N equal to or greater than 3, a first set of points Pᵢ of coordinates (Xᵢ, Yᵢ, Zᵢ) in the first reference system, where i is an integer varying from 1 to N, and a second set of points P′ᵢ of coordinates X′ᵢ, Y′ᵢ, Z′ᵢ) in the second reference system, where i is an integer varying from 1 to N, are considered. Points Pᵢ may correspond to the first previously-described reference points and points P′ᵢ may correspond to the third previously-described points.

The transformation for passing from the first reference system to the second reference system is given by matrixes R and T which verifies the following relationship (1):

P′ᵢ=R.Pᵢ+T+Nᵢ (1)

where R is a rotation matrix, T is a translation vector, and Nᵢ is a noise vector. Matrix R and vector T are determined to minimize criterion S defined according to the following relationship (2):

S=Σᵢ₌₁^{N}∥P′ᵢ−(R, Pᵢ+T)∥² (2)

Call P the center of gravity of points Pᵢ, P′ the center of gravity of points P′ᵢ, points Qᵢ of coordinates (qᵢₓ, q_{iy}, q_{iz}) in the first reference frame given by Qᵢ=Pᵢ−P for i varying from 1 to N, and points Q′ᵢ of coordinates (q′ᵢₓ, q′_{iy}, q′_{iz}) in the second reference frame given by Q′ᵢ=P′ᵢ−P′ for i varying from 1 to N. Relationship (2) then becomes the following relationship (3):

S=Σᵢ₌₁^{N}∥Q′ᵢ−R, Qᵢ∥² (3)

Covariance matrix H according to the following relationship (4) is used:

$$H = {\sum }_{i} [ \begin{matrix}{q}_{ix} \cdot {q}_{ix}^{′} & {q}_{ix} \cdot {q}_{iy}^{′} & {q}_{ix} \cdot {q}_{iz}^{′} \\ {q}_{iy} \cdot {q}_{ix}^{′} & {q}_{iy} \cdot {q}_{iy}^{′} & {q}_{iy} \cdot {q}_{iz}^{′} \\ {q}_{iz} \cdot {q}_{ix}^{′} & {q}_{iz} \cdot {q}_{iy}^{′} & {q}_{iz} \cdot {q}_{iz}^{′}\end{matrix} ] \qquad ( 4 )$$

Matrix H is decomposed into singular values U and V according to the following relationship (5):

H=U A V^{t} (5)

Matrix R and vector T are given by the following relationships (6):

R=V, U^{t} (6)

T=P′−R, P

A point B having its coordinates expressed in the first reference system has a corresponding point B′ having its coordinates expressed in the second reference system. Points B and B′ are connected by the following relationships (7):

B′ᵢ=R.Bᵢ+T (7)

Bᵢ=R⁻¹.B′ᵢ−R⁻¹.T

An advantage of the method using fiducial device 91 is that the transformation can be determined with a better accuracy than on use of a single fiducial element 1, 30, 40, 50, 60, 70, 80, or 81.

The method carries on at step 130.

At step 130, the practitioner determines the position of the implants. To achieve this, the practitioner uses both the three-dimensional internal model and the three-dimensional external model. As an example, on the display screen of interface unit 114, the three-dimensional external model and the three-dimensional internal model may be simultaneously shown in a same image, the three-dimensional external model being correctly positioned with respect to the three-dimensional internal model. The three-dimensional external model is for example added by transparency on the three-dimensional internal model. The practitioner can see in a same image both the bone system of the patient's jaw where the implants should be fixed, and the external surface of the soft tissues, particularly of the gums, covering the bone system. The practitioner can then add to the internal and three-dimensional external models, via interface 114, the three-dimensional model of the external surface of virtual teeth. For each implant, the practitioner can then determine the ideal drilling axis for the fitting of the implant based on the three-dimensional external model completed with the virtual teeth. The axis can be transferred onto the three-dimensional internal model by using the transfer matrix. The practitioner can then adjust the position of the implant axis according to the bone structure of the patient's jaw.

In the case where a surgical guide is to be formed, the practitioner may further determine, based on the three-dimensional internal model and with the position of the implant axes, a three-dimensional model of a stent having its basal surface corresponding to the three-dimensional external model. Further, the practitioner may add, on the three-dimensional model of the stent, the openings necessary for the passage of the drilling tool from the position of the implant axes. Processing unit 112 can then transmit the three-dimensional model of the stent to computer-assisted manufacturing tool 120 for the manufacturing of the surgical guide. The surgical guide can be manufactured by three-dimensional machining methods or additive manufacturing methods, for example, by selective laser fusion, by selective laser sintering, by 3D printing, or by stereolithography.

Generally, the embodiment of the previously-described method of preparing the fitting of dental implants according to the invention can be implemented with any fiducial element which may be repeated both in the three-dimensional external model and the three-dimensional internal model.

FIG. 16 shows a block diagram illustrating a variation of the embodiment of the method of preparing the fitting of dental implants according to the invention in relation with FIG. 15 where step 124 comprises the following sub-steps 150, 152, and 154:

At step 150, a first three-dimensional external model of the teeth and of the gums of the lower or upper portion of the patient's mouth where a prosthesis should be placed is determined, while a first fiducial element 1 or a first fiducial device 91 is fixed to a tooth, to a plurality of teeth, or to the gum of this portion of the mouth, as previously described. The method carries on at step 152.

At step 152, a second additional three-dimensional external model of the teeth and of the gums of the other lower or upper portion of the patient's mouth is determined, while a second fiducial element 1 or a second fiducial device 91 is fixed to a tooth, to a plurality of teeth, or to the gum of this other portion of the mouth. The method carries on at step 154.

At step 154, a third three-dimensional external model of the teeth and of the gums is determined while the two lower and upper portions of the patient's mouth are in occlusion, the first and second fiducial elements 1 or fiducial devices 91 being present at the same positions than on determination of the first and second three-dimensional external models. The third three-dimensional external model is necessarily incomplete since the patient's mouth is in occlusion. However, the third three-dimensional external model enables to know the relative position between the first and second fiducial elements 1 or fiducial devices 91 when the patient's mouth is in occlusion. Processing unit 112 is then capable of placing the first three-dimensional model with respect to the second three-dimensional model when the patient's mouth is in occlusion.

Thereby, at step 130, the practitioner may take into account, by adding virtual teeth to the first three-dimensional external model, constraints determined from the study of the first and second three-dimensional external models in occlusion.

FIG. 17 shows a block diagram illustrating a variation of the embodiment of the method of preparing the fitting of dental implants according to the invention previously described in relation with FIG. 15 adapted to the case where the patient no longer has teeth at the level of the maxillary where an implant is to be placed and uses a denture. According to this variation, previously-described step 124 comprises the following sub-steps 156 and 158:

At step 156, a first three-dimensional external model of the inside of the mouth is determined in the presence of the denture, while a first fiducial element 1 or a first fiducial device 91 is fixed to this denture by gluing, for example. The first three-dimensional external model comprises the external surface of the false teeth of the denture. The method carries on at step 158.

At step 158, a second three-dimensional model of the external surface of the patient's denture is determined while the patient no longer has the denture in his/her mouth and the denture is outside of the patient's mouth. The aim of acquiring this second three-dimensional external model is to have the basal surface of the denture corresponding to the patient's gum surface. The first and second three-dimensional external models are linked due to fiducial element 1 or to fiducial device 91 which enables to have a three-dimensional reference system in relation with the basal surface and a three-dimensional reference system in relation with the external surface of the false teeth.

At step 128, processing unit 112 links the first three-dimensional external model delivered at step 156, the second three-dimensional external model delivered at step 158, and the three-dimensional internal model delivered at step 126.

At previously-described step 130, the practitioner may use the first three-dimensional model of the external surface of the patient's denture as a starting point to adapt the position of the implant axes based on the three-dimensional internal model. In the case where a surgical guide is to be formed, the practitioner uses the second three-dimensional external model which corresponds to the surface (called basal surface) of the surgical guide intended to be in contact with the gums and possibly the palate of the patient.

Advantageously, the portion of fiducial element 1, 40, 50, 60, 70, 80, 81 which enables to recognize the fiducial element in the three-dimensional external model, that is, fiducial faces 12, 14, 16, 34, 36, 38, 72, 82, 84, 89, and the portion of fiducial element 1, 40, 50, 60, 70, 80, 81 which enables to recognize the fiducial element in the three-dimensional internal model, that is, radio-opaque inserts 30, 42, 44, 46, 52, 54, are different. Indeed, the recognition of the fiducial element in the three-dimensional external model advantageously implements surface recognition algorithms while the recognition of the fiducial element in the three-dimensional internal model advantageously uses volume recognition algorithms. This is advantageous since the accuracy of the images capable of being obtained by an X-ray scanner is generally poorer than the accuracy of the images capable of being obtained by an optical camera or a touch probe. The implementation of volume recognition algorithms enables to provide a more robust determination of the reference system associated with the three-dimensional internal model despite the inaccuracy of the images obtained by an X-ray scanner.

The shape of the portion of fiducial element 1, 40, 50, 60, 70, 80, 81 which enables to recognize the fiducial element in the three-dimensional external model can thus be optimized for surface recognition algorithms and the shape of the portion of fiducial element 1, 40, 50, 60, 70, 80 which enables to recognize the fiducial element in the three-dimensional internal model can thus be optimized for volume recognition algorithms.

However, should the accuracy of the images obtained by an X-ray scanner enable it, the determination of the reference system associated with the three-dimensional external model may be performed by surface recognition algorithms based on faces characteristic of the insert.

It may further be advantageous for the entire fiducial element not to be made of a radio-opaque material. Indeed, if the entire fiducial element was made of radio-opaque material, this would risk generating artifacts in the images acquired by the optical camera. Further, if the entire fiducial element was made of a radio-opaque material, it would be difficult to both locate the radio-opaque element under the teeth/gum line to avoid obtaining artifacts on the images acquired by the CT scanner and place at least certain fiducial faces close to the occlusal plane to facilitate the acquisition of images by the intraoral camera.

However, in specific case of total edentulism, the entire fiducial element may be made of radio-opaque material, since risks of artifacts in the images acquired by the CT scanner are decreased.

According to the embodiment of the preparation method previously described in relation with FIG. 15, it is not necessary to form a plaster copy of the patient's mouth to manufacture the drilling guide. Further the steps of image acquisition by camera 116 and of image acquisition by scanner 118 may be performed in a single session which cannot last longer than 20 minutes. The time period between the image acquisition and the manufacturing of the surgical guide may further be decreased, for example, to less than half a day.

Fiducial element 1, 40, 50, 60, 70, 80, or 81 may advantageously be directly fixed to the lateral wall of a tooth or on the gum of the patient. It is thus not necessary to provide a stent which at least partially covers the patient's teeth and which would have the fiducial element fixed thereto.

Specific embodiments of the present invention have been described. Various alterations and modifications will occur to those skilled in the art. In particular, although the fiducial element shown in FIG. 4 has been described with radio-opaque inserts 52, 54 of tubular shape, it should be clear that each tube 52, 54 may be replaced with an element of conical or tapered shape, possibly hollow. Further, although embodiments where the fiducial element is temporarily glued to a patient's tooth or gum have been described, it should be clear that the fiducial element may be fixed by any means in the patient's oral cavity on acquisition of the images with an X-ray scanner and of the images by the optical camera. As an example, the fiducial element may be fixed to the teeth by a mechanical hold system, for example, pliers. Further, even though in the previously-described embodiments, radio-opaque inserts 30, 42, 44, 46, 52, 54 are totally embedded in the block, and particularly covered with the fiducial faces, it should be clear that a portion of the radio-opaque insert may project in an outgrowth out of the block.

Various embodiments with different variations have been described hereabove. It should be noted that those skilled in the art may combine various elements of these various embodiments and variations without showing any inventive step. As an example, the radio-opaque inserts, such as parallelepiped 30 shown in FIG. 1, tubes 52, 54 shown in FIGS. 4 and 7, spheres 42, 44, 46 shown in FIGS. 3 and 9, may be used with any of fiducial elements 1, 60, 70, 80, and 81.

1. A method of preparing the fitting of at least one dental implant, comprising the steps of:
fixing at least one fiducial element to at least a portion of a patient's oral cavity;
acquiring, by means of an optical sensor or of a touch probe, data relative to said at least a portion of the oral cavity;
delivering a first three-dimensional model of the external surface of said at least a portion of the oral cavity based on data relative to said at least a portion of the oral cavity;
acquiring, by means of an X-ray scanner, data relative to at least a portion of the bone system of the patient's jaw;
delivering a second three-dimensional model of said at least a portion of the bone system of the patient's jaw based on data relative to said at least a portion of the bone system of the jaw;
performing a computer recognition of faces of said at least one fiducial element in the first model, said faces being made of a material transparent to X rays;
determining by computer means a first three-dimensional coordinate system associated with the first model;
performing a computer recognition of at least a portion locatable with X rays of said at least one fiducial element in the second model;
determining by computer means a second coordinate system associated with the second model;
determining by computer means a transformation rela-tionship for passing from the first coordinate system to the second coordinate system based on said faces and on said portion; and
determining the position of said implant based on the first model and on the second model and based on the first three-dimensional coordinate system, on the second coordinate system, and on the transformation relationship.

2. The method of claim 1, wherein the step of determining the position of said implant based on the first model and on the second model and based on the first coordinate system and on the second coordinate system comprises the steps of:
determining the position of a dental prosthesis associated with said implant in the first model;
determining the theoretical position of the implant in the second model based on the position of the associated dental prosthesis in the first model; and
determining the ideal position of the implant in the second model based on the theoretical position.

3. The method of claim 1, further comprising the steps of:
fixing an additional fiducial element to another portion of the oral cavity opposite to said portion;
acquiring, by means of the optical sensor or of the touch probe, data relative to said other portion of the oral cavity;
delivering a three-dimensional model of the external surface of said other portion of the oral cavity based on data relative to said other portion of the oral cavity;
acquiring, by means of the optical sensor or of the touch probe, data relative to the fiducial element and to the additional fiducial element when the mouth is in occlusion; and
delivering a three-dimensional model of the external surface of the fiducial element and of the additional fiducial element when the mouth is in occlusion.

4. The method of claim 3, wherein the step of determining the position of said implant is carried out based on the first model and on the three-dimensional model of the external surface of said other portion of the oral cavity placed in occlusion.

5. The method of claim 1, wherein a denture is capable of being placed on said portion of the oral cavity, wherein the first model is determined in the absence of the denture, the method further comprising the steps of:
arranging the denture on said portion of the oral cavity;
acquiring, by means of the optical sensor or of the touch probe, data relative to the denture; and
delivering a three-dimensional model of the external surface of the denture based on the data relative to the denture.

6. The method of claim 1, wherein the method of determining the second coordinate system comprises determining the inertia matrix of said portion.

7. The method of claim 1, further comprising determining the drilling axis of said implant, determining a three-dimensional model of a stent comprising a cylindrical opening along the drilling axis of said implant, and manufacturing in computer-assisted fashion said stent comprising said opening.

8. The method of claim 1, further comprising the steps of:
fixing at least three fiducial elements to said portion of a patient's oral cavity;
performing a computer recognition of faces of each fiducial element in the first model;
determining by computer means, for each fiducial element, a first reference point in the first model;
performing a computer recognition of at least a portion of each fiducial element in the second model;
determining by computer means, for each fiducial element, a second reference point in the second model; and
determining by computer means the relationship for passing from the first coordinate system to the second coordinate system based on the first three reference points and on the second three reference points

9. A system for preparing the fitting of dental implants, the system comprising:
at least one fiducial element comprising at least three non-parallel faces visible with an optical camera and/or a touch probe and at least a portion locatable with X rays, said faces being made of a material transparent to X rays, said fiducial element being capable of being fixed to at least a portion of a patient's oral cavity;
an optical image sensor or a touch probe capable of acquiring data relative to said at least a portion of the oral cavity;
an X-ray scanner capable of acquiring data relative to at least a portion of the bone system of the patient's jaw; and
a processing unit connected to the X-ray scanner and to the optical image sensor and/or to the touch probe, the processing unit, the optical image sensor, and/or the X-ray scanner being capable of delivering a first three-dimensional model of the external surface of said at least a portion of the oral cavity based on the data relative to said at least a portion of the oral cavity, delivering a second three-dimensional model of said at least a portion of the bone system of the patient's jaw based on the data relative to said at least a portion of the bone system of the jaw, recognizing the faces in the first model, determining a first three-dimensional coordinate system associated with the first model, recognizing said at least a portion in the second model, determining a second coordinate system associated with the second model, determining a transformation relationship for passing from the first coordinate system to the second coordinate system based on said faces and on said portion, and determining the position of said implant based on the first model and on the second model and based on the first three-dimensional coordinate system, on the second coordinate system, and on the transformation relationship.

10. The system of claim 9, wherein the material transparent to X rays is further opaque to visible light.

11. The system of claim 9, wherein at least two faces from among said three faces are planar and inclined with respect to each other by an angle in the range from 5° to 85° or from 95° to 270°.

12. The system of claims 9, wherein at least one of the faces corresponds to a portion of a sphere or to a portion of a cylinder.

13. The system of claim 9, wherein said portion is covered with said faces.

14. The system of claim 9, wherein said portion comprises at least two rectilinear non-intersecting tubes.

15. The system of claim 9, wherein said portion comprises at least three spheres having non-aligned centers.

16. The system of claim 15, wherein the spheres have different diameters.

17. The system of claim 9, wherein said portion comprises at least one parallelepiped.

18. The system of claim 9, wherein said at least one fiducial element comprises at least first, second, and third planar non-parallel faces, at least the first face being inclined with respect to the second face by an angle in the range from 5° to 85° or from 95° to 270° and the first face (12) being inclined with respect to the third face by an angle in the range from 5° to 85° or from 95° to 270°.

19. The system of claim 9, comprising at least three distinct fiducial elements each comprising at least three non-parallel faces visible with the optical camera and/or the touch probe and at least a portion locatable with X rays, each fiducial element being capable of being fixed to at least a portion of a patient's oral cavity.
